(19) United States
(12) Patent Application Publication
(10) Pub. No.: US 2011/0124700 A1
(43) Pub. Date: May 26, 2011

(54) SYSTEMS AND METHODS OF CANCER STAGING AND TREATMENT

(76) Inventor: Glen Weiss, Phoenix, AZ (US)

(21) Appl. No.: 12/735,866

(22) PCT Filed: Feb. 19, 2009
(86) PCT No.: PCT/US2009/001046

§ 371 Date: Dec. 2, 2010

Related U.S. Application Data
(60) Provisional application No. 61/029,656, filed on Feb. 19, 2008.

(51) Int. Cl. A61K 31/4045; C12Q 1/68; C40B 30/04; A61P 35/00

(52) U.S. Cl. 514/414; 435/6; 506/9

(57) ABSTRACT

Methods of assessing the sensitivity of a cancer cell to a tyrosine kinase inhibitor are disclosed. Such methods include assessing the expression of miR-497 and correlating reduced expression with sensitivity to the tyrosine kinase inhibitor. Also disclosed are methods of assessing the sensitivity of a cell to a tyrosine kinase inhibitor that includes assessing the expression of FGF1, HOXC10, and/or LHFP. Additionally disclosed are methods of treating patients with tyrosine kinase inhibitors such as sunitinib based on results obtained from the disclosed methods and kits that facilitate the methods.

### RELATED APPLICATIONS

This application claims priority to U.S. Provisional Application 61/029,656, filed 19 Feb. 2008, the content of which is hereby incorporated by reference in its entirety.

### FIELD

Provided herein are methods of treatment of cancer and more specifically methods of the differential treatment of cancer based upon microRNA gene expression.

### BACKGROUND

Worldwide, lung cancer is the leading cause of cancer-related mortality in both men and women. Although current treatments for advanced non-small cell lung cancer (NSCLC) are disappointing, there is growing promise using anti-angiogenic therapies. Some of these anti-angiogenic therapies, which include tyrosine kinase inhibitors (TKI) targeting VEGF receptor 2 and 3 (VEGFR-2/3), such as sunitinib and sorafenib, have been administered to patients in an unselected fashion.

MicroRNAs (also known as miRs or miRNAs) are a class of small non-coding RNAs having 21 to 25 nucleotides in length that have recently been implicated in cancer biology (Calin et al., Proc Natl Acad Sci USA 99:15524-15529, 2002). These RNA fragments post-transcriptionally regulate gene expression by binding to complementary sequences in the 3′ untranslated region (3′UTR) of the target mRNA (Kumar et al., Nat Genet 39:673-677, 2007). This can ultimately lead to repression of protein translation and, as a result, of protein expression (Eder et al., N Eng J Med 352:2446-2448, 2005). Regulation of protein expression by miRs is emerging as an important area of study in carcinogenesis because their regulatory capabilities can drastically influence cell physiology (Scott et al., J Biol Chem 282:1479-1486, 2007). Therefore, there is a need for methods of predicting drug efficacy in individual patients by miR expression.

### BRIEF SUMMARY

In one aspect, provided is a method of evaluating the sensitivity of a tumor to a tyrosine kinase inhibitor using microRNA.

In one embodiment, provided is a method of evaluating a cancer cell sensitivity to a tyrosine kinase inhibitor by assessing the expression of miR-497 in a patient sample and correlating a reduced expression of the miR-497 with a sensitivity to the tyrosine kinase inhibitor.

In another embodiment, provided is a method of predicting a cancer cell sensitivity to a tyrosine kinase inhibitor in order to apply a personalized medicine based therapy to cancer treatment.

In yet another embodiment, provided is a method of predicting a cancer cell sensitivity to a tyrosine kinased inhibitor in order to prevent a patent from suffering side effects of a drug that is unlikely to be efficacious.

In another embodiment, provided is a method of predicting a cancer cell sensitivity to a tyrosine kinase inhibitor in order to prevent a cancer patient from wasting time on a treatment that is unlikely to be efficacious.

In another embodiment, provided is a test that evaluates a sensitivity of a tumor to a tyrosine kinase inhibitor that does not require a tumor biopsy.

In one embodiment, a patient sample comes from blood. In another embodiment, a patient sample comes from a tumor biopsy.

In another embodiment, an expression is assessed by mRNA detection methods such as RTPCR, microarray analysis, or Northern blot.

In another embodiment, a tyrosine kinase inhibitor specifically inhibits VEGFR2 alone, VEGFR3 alone, both VEGFR2 and VEGFR3, or either VEGFR2 or VEGFR3 in combination with any other molecule. In one embodiment, a tyrosine kinase inhibitor is sunitinib. In another embodiment, a tyrosine kinase inhibitor is sorafenib.

In yet another embodiment, the cancer cell is a non-small cell lung cancer cell.

In another aspect, provided is a method involving slowing the expansion of a population of cancer cells including assessing the expression of miR-497 in a sample, correlating reduced expression of the miR-497 with sensitivity to a tyrosine kinase inhibitor, and treating the population of cancer cells with the tyrosine kinase inhibitor

In one embodiment, provided is a method of correlating positive expression of miR-497 with resistance to a tyrosine kinase inhibitor. In this case, the tyrosine kinase inhibitor is not administered.

In one embodiment, a sample is taken from a human. In another embodiment, the sample is a blood fraction. In yet nother embodiment, the sample is a tumor biopsy.

In one embodiment, cancer cells are shown to display a loss of heterozygosity in chromosomal region 17p.

In one embodiment, cancer cells are non-small cell lung cancer cells.

In one embodiment, a tyrosine kinase inhibitor specifically inhibits VEGFR2 alone, VEGFR3 alone, both VEGFR2 and VEGFR3, or either VEGFR2 or VEGFR3 in combination with any other molecule.

In another embodiment, a tyrosine kinase inhibitor is sunitinib.

In yet another embodiment, the population of cancer cells comprises non-small cell lung cancer cells.

In yet another aspect, provided are kits that facilitate the assessment of the expression of miR-497 that includes a reagent that is capable of specifically recognizing the miR-497 itself or a product of the miR-497 gene. In one embodiment, the reagent comprises an oligonucleotide. In another embodiment, the reagent comprises an antisense nucleic acid. In yet another embodiment, the reagent is bound to a solid support.

In one embodiment, the kit contains a fluorescent label. In another embodiment, the kit contains a reagent capable of recognizing a gene other than the miR-497 gene or any product of that gene.

In another aspect, provided herein is a method involving an evaluation of cancer cells sensitivity to a tyrosine kinase inhibitor by assessing the expression of FGF1, HOXC10, and/or LHFP, whether singly or in combination, and correlating the positive expression of these products with sensitivity to the tyrosine kinase inhibitor.

In one embodiment, a sample comes from a patient's blood. In another embodiment, a sample comes from a tumor biopsy.

In one embodiment, an expression is assessed by measuring a mRNA expression using methods such as RTPCR, microarray analysis, or Northern blot. In another embodiment, an expression is assessed by measuring a protein expression using methods that involve specific ligands such as antibodies. Such methods include immunohistochemical methods, ELISA, and flow cytometry. In yet another embodiment, an expression is assessed using the methods of mass spectrometry.

In one embodiment, a tyrosine kinase inhibitor is sunitinib.

In another embodiment, a cancer cell is a non-small cell lung cancer cell.

In another aspect, provided herein is a method involving slowing the expansion of a population of cancer cells comprising assessing the expression of FGF1, HOXC10, and/or LHFP, alone or in combination in a sample, correlating positive expression of FGF1, HOXC10, and/or LHFP with sensitivity to the tyrosine kinase inhibitor, and treating the population of cancer cells with the tyrosine kinase inhibitor.

In one embodiment, provided is a method of correlating reduced expression of FGF1, HOXC10, and/or LHFP with resistance to the tyrosine kinase inhibitor. In this case, the tyrosine kinase inhibitor is not administered.

In one embodiment, a sample is taken from a human. In another embodiment, a sample is a blood fraction. In another embodiment, a sample is a tumor biopsy. In yet another embodiment, cancer cells are non-small cell lung cancer cells. In another embodiment, the tyrosine kinase inhibitor is sunitinib.

In yet another aspect, provided are kits that facilitate the assessment of the expression of FGF1, HOXC10, and/or LHFP, alone or in combination, that include a reagent that is capable of specifically recognizing FGF1, HOXC10, and/or LHFP themselves or a product of the FGF1, HOXC10, and/or LHFP genes. In one embodiment, the reagent comprises an oligonucleotide. In another embodiment, the reagent comprises an antisense nucleic acid. In yet another embodiment, the reagent is bound to a solid support. In another embodiment, the kit contains a fluorescent label. In another embodiment, the kit contains a reagent capable of recognizing a gene other than the miR-497 gene or any product of that gene.

### BRIEF DESCRIPTION OF THE DRAWINGS

A more complete understanding of the present invention may be derived by referring to the detailed description when considered in connection with the following illustrative figures. In the figures, like reference numbers refer to like elements or acts throughout the figures.

FIG. 1 depicts a Western blot image showing expression of VEGFR2 and VEGFR3 in H358 cells transfected with miR-497 mimic or control mimic microRNA.

FIG. 2 depicts the results of densitometry of Western blots showing expression of VEGFR2 and VEGFR3 in H358 cells transfected with miR-497 inhibitor or mimic or control inhibitor or mimic microRNA.

FIG. 3 depicts the results of densitometry of Western blots showing expression of VEGFR2 in H1703 cells transfected with miR-497 inhibitor or mimic or control inhibitor or mimic microRNA.

FIG. 4 depicts the results of densitometry of Western blots showing expression of VEGFR2 in H520 cells transfected with miR-497 inhibitor or mimic or control inhibitor or mimic microRNA.

FIG. 5 depicts the results of densitometry of Western blots showing expression of VEGFR2 in H157 cells transfected with miR-497 inhibitor or mimic or control inhibitor or mimic microRNA.

FIG. 6 depicts the results of densitometry of Western blots showing expression of VEGFR2 in H1703 cells transfected with miR-497 inhibitor or mimic or control inhibitor or mimic microRNA.

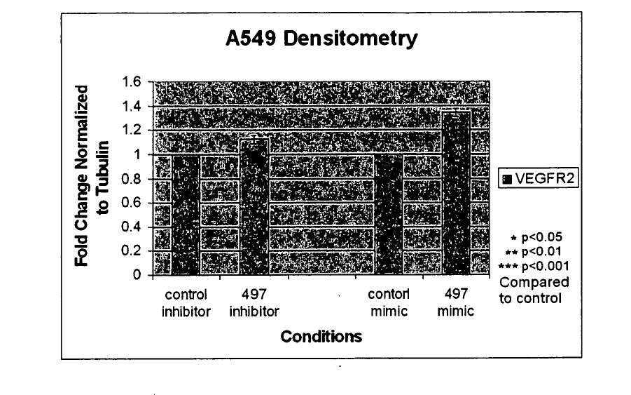

FIG. 7 depicts the results of densitometry of Western blots showing expression of VEGFR2 and VEGFR3 in H1703 cells transfected with miR-497 inhibitor or mimic or control inhibitor or mimic microRNA.

FIG. 8 depicts the results of sunitinib treatment on the viability of sunitinib sensitive (H520 and H1703) and sunitinib resistant (H322c, H358, H157, and A549) cell lines.

FIG. 9 depicts the results of qRT-PCR analysis of FGF1, LHFP, and HOXC10 in sunitinib sensitive (H520 and H1703) and sunitinib resistant (H322c, H358, H157, and A549) cell lines.

### DETAILED DESCRIPTION

Definitions

Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth in order to provide a thorough understanding of the various aspects of the invention. It will be understood, however, by those skilled in the relevant arts, that the present invention may be practiced without these specific details. In other instances, known structures and devices are shown or discussed more generally in order to avoid obscuring the invention. The full scope of the disclosure is not limited to the examples that are described below.

Methods of Use

Provided are methods of assessing the sensitivity of cancer cells to a drug using an miR-497 mimic in combination with a tyrosine kinase inhibitor. Also provided are methods of treating cancer with a tyrosine kinase inhibitor based upon the expression of miR-497 by the cancer cells. Additionally, provided are methods of assessing the sensitivity of cancer cells to a tyrosine kinase inhibitor on the basis of the expression of any of the genes FGF1, HOXC10, and LHFP, whether singly or in combination.

A target includes any molecular structure produced by a cell and expressed inside the cell, on the cell surface, or secreted by the cell. Targets include proteins, lipids, carbohydrates, nucleic acids, including RNA molecules and genomic DNA sequences, subcellular structures, glycoproteins, viruses and any other like structures known or yet to be disclosed whether alone or in combination. Illustrative examples of targets include, but are not limited to, VEGFR2, VEGFR3, miR-497, FGF1, HOXC10, LHFP and any products thereof including mRNA's and proteins.

Cancer cells include any cells derived from a tumor, neoplasm, cancer, precancer, cell line, or any other source of cells that have the potential to expand and grow to an unlimited degree. Cancer cells are derived from naturally occurring sources or are artificially created. Cancer cells are capable of invasion into other tissues and metastasis when placed into an animal host. Cancer cells further encompass any malignant cells that have invaded other tissues and/or metastasized. One or more cancer cells in the context of an organism may also be called a cancer, tumor, neoplasm, growth, malignancy, or any other term used in the art to describe cells in a cancerous state.

Cancers that serve as sources of cancer cells include, but are not limited to, solid tumors such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon cancer, colorectal cancer, kidney cancer, pancreatic cancer, bone cancer, breast cancer, ovarian cancer, prostate cancer, esophageal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular cancer, small cell lung carcinoma, bladder carcinoma, lung cancer, epithelial carcinoma, glioma, glioblastoma multiforme, astrocytoma, medulloblastoma, craniopharyngioma, ependymoma, pinealoma, hemangioblastoma, acoustic neuroma, oligodendroglioma, meningioma, skin cancer, melanoma, neuroblastoma, and retinoblastoma.

Additional cancers that serve as sources of cancer cells include, but are not limited to, blood borne cancers such as acute lymphoblastic leukemia, acute lymphoblastic B-cell leukemia, acute lymphoblastic T-cell leukemia, acute myeloblastic leukemia, acute promyelocytic leukemia, acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia, chronic lymphocytic leukemia, hairy cell leukemia, multiple myeloma, lymphoblastic leukemia, myelogenous leukemia, lymphocytic leukemia, myelocytic leukemia, Hodgkin's disease, non-Hodgkin's lymphoma, Waldenstrom's macroglobulinemia, heavy chain disease, and polycythemia vera.

In one embodiment, cancer cells are derived from non-small cell lung cancer (NSCLC.) NSCLC includes any carcinoma derived from lung tissues that does not include small cell lung cancers. Examples of non-small cell lung cancers include, but are not limited to, adenocarcenomas, large cell carcinomas, and squamous cell carcinomas of the lung.

Expansion of a cancer cell includes any process that results in an increase in the number of individual cells derived from a cancer cell. Expansion of a cancer cell may result from mitotic division, proliferation, or any other form of expansion of a cancer cell, whether in vitro or in vivo. Expansion of a cancer cell further encompasses invasion and metastasis. A cancer cell may be in physical proximity to cancer cells from the same clone or from different clones that may or may not be genetically identical to it. Such aggregations may take the form of a colony, tumor or metastasis, any of which may occur in vivo or in vitro. Slowing the expansion of the cancer cell may be brought about either by inhibiting cellular processes that promote expansion or by bringing about cellular processes that inhibit expansion. Processes that inhibit expansion include processes that slow mitotic division and processes that promote cell senescence or cell death. Examples of specific processes that inhibit expansion include caspase dependent and independent pathways, autophagy, necrosis, apoptosis, and mitochondrial dependent and independent processes and further include any such processes yet to be disclosed.

Inhibition of the expansion of a cancer cell is achieved through the use of an outside agent applied to a cancer cell for the purpose of slowing the expansion of a cancer cell. Such agents include natural or synthetic ligands, blockers, agonists, antagonists or activators of receptors, immune cells, such as CD8+ T cells, viruses, inhibitors of gene or protein expression, such as siRNA or miR's, small molecules, pharmaceutical compositions, or any other composition of matter that when administered to a cancer cell results in slowing of the expansion of a cancer cell. The concept of agents that slow the expansion of a cancer cell encompasses restricting access to any natural or artificial agent necessary for cell survival including necessary nutrients, ligands, or cell-cell contacts. Examples of such agents and conditions include treatment with antiangiogenic inhibitors.

In one embodiment, an agent that slows the expansion of a cancer cell comprises a tyrosine kinase inhibitor (TKI). A tyrosine kinase catalyzes the transfer of a phosphate group to the tyrosine residue of a specific protein. If a TKI inhibits an action of a kinase necessary for growth, differentiation or division of a cancer cell, expansion of a cancer cell is slowed. A TKI includes any agent that inhibits the action of one or more tyrosine kinases in a specific or non-specific fashion. TKIs include small molecules, antibodies, peptides, or anything that directly, indirectly, allosterically, or in any other way inhibits tyrosine residue phosphorylation.

Specific examples of tyrosine kinase inhibitors include N-(trifluoromethylphenyl)-5-methylisoxazol-4-carboxamide, 3-[(2,4-dimethylpyrrol-5-yl)methylidenyl)indolin-2-one, 17-(allylamino)-17-demethoxygeldanamycin, 4-(3-chloro-4-fluorophenylamino)-7-methoxy-6-[3-(4-morpholinyl)propoxyl]quinazoline, N-(3-ethynylphenyl)-6,7-bis(2-methoxyethoxy)-4-quinazolinamine, BIBX1382, 2,3,9,10,11,12-hexahydro-10-(hydroxymethyl)-10-hydroxy-9-methyl-9,12-epoxy-1H-diindolo[1,2,3-fg:3′,2′,1′-k1]pyrrolo[3,4-i][1,6]benzodiazocin-1-one, SH268, genistein, STI571, CEP2563, 4-(3-chlorophenylamino)-5,6-dimethyl-7H-pyrrolo [2,3-d]pyrimidinemethane sulfonate, 4-(3-bromo-4-hydroxyphenyl)amino-6,7-dimethoxyquinazoline, 4-(4′-hydroxyphenyl)amino-6,7-dimethoxyquinazoline, SU6668, STI571A, N-4-chlorophenyl-4-(4-pyridylmethyl)-1-phthalazinamine, N-[2-(diethylamino)ethyl]-5-[(Z)-(5-fluoro-1,2-dihydro-2-oxo-3H-indol-3-ylidine)methyl]-2,4-dimethyl-1H-pyrrole-3-carboxamide (sunitinib), 4-[4-[[4-chloro-3 (trifluoromethyl)phenyl]carbamoylamino] phenoxy]-N-methyl-pyridine-2-carboxamide (sorafenib), and EMD121974.

In another embodiment, a tyrosine kinase inhibitor has activity upon Vascular Endothelial Growth Factor Receptor 2 (VEGFR2) and Vascular Endothelial Growth Factor Receptor 3 (VEGFR3). VEGFR2 and VEGFR3 are tyrosine kinases that phosphorylate proteins necessary for angiogenesis. Tyrosine kinase inhibitor may inhibit either VEGFR2 or VEGFR3 singly, both VEGFR2 and VEGFR3 to the exclusion of all other targets, or VEGFR2 and/or VEGFR3 in combination with one or more additional tyrosine kinases or other targets. Tyrosine kinase inhibitors that inhibit VEGFR2 and/or VEGFR3 include sunitinib and sorafenib, but could be any inhibitor that targets VEGFR2 singly or in combination with any other tyrosine kinase, any inhibitor that targets VEGFR3 singly or in combination with any other tyrosine kinase, or any inhibitor that targets either VEGFR2 and VEGFR3 to the exclusion of or in combination with any other tyrosine kinase.

As used herein, VEGFR2 has the sequence provided below.

(SEQ ID NO: 2)
gcagcatcgg acaagacccc cagcacttgg gggttcaggc
ccggcagggc gggcagaggg ctggaggccc aggctgggaa
ctcatctggt tgaactctgg tggcacagga gtgtcctctt
ccctctctgc agacttccca gctaggaaga gcaggactcc
aggcccaagg ctcccggaat tccgtcacca cgactggcca
gggccacgct ccagctgccc cggcccctcc ccctgagatt
cagatgtcat ttagttcagc atccgcaggt gctggtcccg
gggccagcac ttccatggga atgtctcttt ggcgacctcc
tttcatcaca ctgggtggtg gcctggtccc tgttttccca
cgaggaatct gtgggtctgg gagtcacaca gtgttggagg
ttaaggcata cgagagcaga ggtctcccaa acgccctttc
ctcctcaggc acacagctac tctccccacg agggctggct
ggcctcaccc acccctgcac agttgaaggg aggggctgtg
tttccatctc aaagaaggca tttgcagggt cctcttctgg
gcctgaccaa acagccaact agcccctggg gtggccacca
gtatgacagt attatacgct ggcaacacag aggcagcccg
cacacctgcg cctgggtgtt gagagccatc ctgcaagtct
ttttcaacag aacttcacag actgttagag ctgctgagaa
gaatttgctt tccgaattca gcctggaagg cgcccaggga
cagctgtact gagtctagat gactctgacc cccgccccag
gtcaaggcca gcagagcagt cagtgcctct ggagaaggcc
cttgctctcc cacctggccc agactccgag gagcctgggt
ctggagctgc cggtctggtt cttcccttta gagcccggat
ctgccacctg cggcccctcc caagccgtga accagctcat
gagagatgaa cactgtggga tccactcagg aaggctcggg
gctggcacaa aggaccaccc agcattgccc tgtgccaccc
agcactcagt ggacattctg gggacctgcc ttcagccttt
tcctgccctg tgcctgacat cagcaccctg gctggtcaga
atgccgccct cccagaggag cagccgagag atcccctgaa
ggctggaggc attctgctca ggacccctat cccagctcac
agtgcccaac catctcacca ggagaaagag ccacatcccc
acgttaggac cacggagact gaccaccacc ctgacccccc
aaacccacgc accagacgct tgcaggacag gcgccgcgca
gcgggcaggg gcttgcccgg ccgaccctcc cctccccacc
tcccccactg cgcgttactc caggatatgc cgagtgcacg
tataaggtca tcttcgtcgt ccccgtggac ctcccccttc
ctctgcacgt cgtccaacgt gggactggcg tgtcaggctt
ccctgggagg atctggaggt tgttctctgc agagaaccag
cctggctcct ggcgcgcacc tctgctccct tctcctcact
acccacccac gcatgtaccg ggaaaaaaac tactatgccc
ttctagacca tgttctgaga aaagatcgaa aatatttaac
aagagataat aataaatctg atgccggtct ttgtgtgtgt
tgcgga

VEGFR3 has the sequence provided below.

(SEQ ID NO: 3)
gcagcatcgg acaagacccc cagcacttgg gggttcaggc
ccggcagggc gggcagaggg ctggaggccc aggctgggaa
ctcatctggt tgaactctgg tggcacagga gtgtcctctt
ccctctctgc agacttccca gctaggaaga gcaggactcc
aggcccaagg ctcccggaat tccgtcacca cgactggcca
gggccacgct ccagctgccc cggcccctcc ccctgagatt
cagatgtcat ttagttcagc atccgcaggt gctggtcccg
gggccagcac ttccatggga atgtctcttt ggcgacctcc
tttcatcaca ctgggtggtg gcctggtccc tgttttccca
cgaggaatct gtgggtctgg gagtcacaca gtgttggagg
ttaaggcata cgagagcaga ggtctcccaa acgccctttc
ctcctcaggc acacagctac tctccccacg agggctggct
ggcctcaccc acccctgcac agttgaaggg aggggctgtg
tttccatctc aaagaaggca tttgcagggt cctcttctgg
gcctgaccaa acagccaact agcccctggg gtggccacca
gtatgacagt attatacgct ggcaacacag aggcagcccg
cacacctgcg cctgggtgtt gagagccatc ctgcaagtct
ttttcaacag aacttcacag actgttagag ctgctgagaa
gaatttgctt tccgaattca gcctggaagg cgcccaggga
cagctgtact gagtctagat gactctgacc cccgccccag
gtcaaggcca gcagagcagt cagtgcctct ggagaaggcc
cttgctctcc cacctggccc agactccgag gagcctgggt
ctggagctgc cggtctggtt cttcccttta gagcccggat
ctgccacctg cggcccctcc caagccgtga accagctcat
gagagatgaa cactgtggga tccactcagg aaggctcggg
gctggcacaa aggaccaccc agcattgccc tgtgccaccc
agcactcagt ggacattctg gggacctgcc ttcagccttt
tcctgccctg tgcctgacat cagcaccctg gctggtcaga
atgccgccct cccagaggag cagccgagag atcccctgaa
ggctggaggc attctgctca ggacccctat cccagctcac
agtgcccaac catctcacca ggagaaagag ccacatcccc
acgttaggac cacggagact gaccaccacc ctgacccccc
aaacccacgc accagacgct tgcaggacag gcgccgcgca
gcgggcaggg gcttgcccgg ccgaccctcc cctccccacc
tcccccactg cgcgttactc caggatatgc cgagtgcacg
tataaggtca tcttcgtcgt ccccgtggac ctcccccttc
ctctgcacgt cgtccaacgt gggactggcg tgtcaggctt
ccctgggagg atctggaggt tgttctctgc agagaaccag
cctggctcct ggcgcgcacc tctgctccct tctcctcact
acccacccac gcatgtaccg ggaaaaaaac tactatgccc
ttctagacca tgttctgaga aaagatcgaa aatatttaac
aagagataat aataaatctg atgccggtct ttgtgtgtgt
tgcgga

However, contemplated is any sequence identifiable as VEGFR2 or VEGFR3 on the basis of its ability to be inhibited by one or more tyrosine kinase inhibitors, its ability to recognize a specific ligand, or its ability to perpetuate an intracellular signal. A sequence may display any one of these characteristics and any combination thereof. A sequence includes any mutation, truncation, or addition of extra nucleotides. A sequence having at least 60% 70%, 80%, or 90% identity to SEQ ID NO. 2 in the case of VEGFR2 and at least 60% 70%, 80%, or 90% identity to SEQ ID NO. 3 in the case of VEGFR3 at the genomic, mRNA, or protein level.

MicroRNA's (miR's) are non-coding RNAs having 18 to 36 nucleotides, in one embodiment 21 to 25 nucleotides in length that inhibit gene expression by binding to a sequence complementary to the miR sequence, often located in the 3′ untranslated region (UTR) of the target mRNA. Mechanisms of gene silencing include repression of protein translation and downregulation of protein expression.

As used herein, miR-497 has the sequence provided below.

| | |
|---|---|
| cagcagcaca cugugguuug u | (SEQ ID NO: 1) |

However, contemplated is any sequence identifiable as miR-497 including mutations, truncations, or additions of one or more nucleotides that is capable of binding the 3′ UTR of VEGFR2 or VEGFR3 with such binding resulting in a reduced VEGFR2 or VEGFR3 expression. A sequence having at least 60%, 70%, 80%, or 90% identity to SEQ ID NO.1 at the genomic or mRNA level. The concept of miR-497 includes one or more non-nucleotide small molecule compositions of matter derived from miR-497 capable of specifically binding to the 3′ UTR of VEGFR2/3 such that VEGFR2/3 expression is silenced.

While a specific target is identified by a nucleic acid sequence, such as a cDNA, mRNA or protein sequence, a specific target is not limited to the products of that exact sequence. Rather, a specific target identified by a nucleic acid sequence encompasses all sequences that, when their expression is assessed, yield positive expression when assessed by the same method as the specific target. In one embodiment, if expression of a specific target in a sample is assessed by immunohistochemical analysis, and if a sample expresses a sequence different from the sequence used to identify the specific target (e.g., a variation of one or more nucleic acid molecules,) but positive expression is still determined, then the specific target encompasses the sequence expressed by the sample.

Expression encompasses all processes through which material derived from a nucleic acid template is produced. Expression thus includes RNA transcription, mRNA splicing, protein translation, protein folding, post-translational modification, membrane transport, associations with other molecules, addition of carbohydrate moeties to proteins, phosphorylation, protein complex formation and any other process along a continuum that results in biological material derived from genetic material. Expression also encompasses all processes through which the production of material derived from a nucleic acid template is actively or passively suppressed. Such processes include all aspects of transcriptional and translational regulation. Examples include heterochromatic silencing, transcription factor inhibition, any form of RNAi silencing, microRNA silencing, alternative splicing, protease digestion, post-translational modification, and alternative protein folding.

Expression is assessed by any number of methods used to detect material derived from a nucleic acid template used currently in the art and yet to be developed. Examples of such methods include any nucleic acid detection method including, but not limited to, microarray analysis, RNA in situ hybridization, RNAse protection assay, Northern blot, reverse transcriptase PCR, quantitative PCR, quantitative reverse transcriptase PCR, quantitative real-time reverse transcriptase PCR, or any other method of detecting a specific nucleic acid known or yet to be disclosed. Other examples include any process of detecting expression that uses an antibody including, but not limited to, flow cytometry, immunohistochemical methods, ELISA, Western blot, and immunoaffinity chromatograpy. Antibodies may be monoclonal, polyclonal, or any antibody fragment including an Fab, F(ab)2, Fv, scFv, phage display antibody, peptibody, multispecific ligand, or any other reagent with specific binding to a target. Such methods also include direct methods used to assess protein expression including, but not limited to HPLC, mass spectrometry, protein microarray analysis, PAGE analysis, isoelectric focusing, 2-D gel electrophoresis, and enzymatic assays. Samples from which expression is detected include single cells, whole organs or any fraction of a whole organ, whether in vitro, ex vivo, in vivo, or post-mortem.

Other methods used to assess expression include the use of natural or artificial ligands capable of specifically binding a target, including a protein, carbohydrate, fat, nucleic acid, catalytic site, or any combination of these such as an enzyme, glycoprotein, cell membrane, virus, cell, organ, organelle, or any other multimolecular structure that constitutes a target that is specifically bound by a ligand. Such ligands include antibodies, antibody complexes, conjugates, natural ligands, small molecules, nanoparticles, or any other molecular entity capable of specific binding to a target. Ligands are associated with a label such as a radioactive isotope or chelate thereof, dye (fluorescent or nonfluorescent,) stain, enzyme, metal, or any other substance capable of aiding a machine or a human eye from differentiating a cell expressing a target from a cell not expressing a target. Additionally, expression may be assessed by monomeric or multimeric ligands associated with substances capable of killing a cell. Such substances include protein or small molecule toxins, cytokines, pro-apoptotic substances, pore forming substances, radioactive isotopes, or any other substance capable of killing a cell.

Positive expression includes any difference between a cell expressing a specific target and a cell that does not express a specific target. The exact nature of positive expression varies by the method, but is well known to those practicing a particular method. Positive expression is assessed by a detector, an instrument containing a detector, or by aided or unaided human eye. Examples include, but are not limited to, specific staining of cells expressing a target in an IHC slide, binding of RNA from a sample to a microarray and detection by an instrument capable of detecting the binding to said microarray, a high rate of dye incorporation in real-time RTPCR, detection of fluorescence on a cell expressing a target by a flow cytometer, the presence of radiolabeled bands on film in a Northern blot, detection of labeled blocked RNA by RNAse protection assay, cell death measured by apoptotic markers, cell death measured by shrinkage of a tumor, or any other method by which expression is observed known or yet to be disclosed.

Reduced expression constitutes a lack of positive expression such that there is not a significant difference between a cell expressing a particular target and a cell not expressing the particular target. The concept of reduced expression further encompasses insufficient expression to reach or exceed a threshold, cutoff, or level that results in a particular cellular or physiological response. For example, reduced expression includes the expression of a particular target in a test cell that is positive expression relative to a control cell known not to express the target. However, because the expression of the target in the test cell is insufficient to cause a particular physiological response (e.g., rendering the cell sensitive to a particular drug), the expression in the test cell is still classified as reduced expression. Similarly, the concept of positive expression also encompasses expression sufficient to cause a physiological response.

Also provided are methods of assessing the expression of a target in any biological sample from which the expression is assessed. One skilled in the art knows how to select a particular biological sample and how to collect said sample depending upon whether or not expression of germline DNA, tumor DNA, mRNA, or any form of protein is assessed. Examples of sources of samples include, but are not limited to, biopsy or other in vivo or ex vivo analysis of prostate, breast, skin, muscle, facia, brain, endometrium, lung, head and neck, pancreas, small intestine, blood, liver, testes, ovaries, colon, skin, stomach, esophagus, spleen, lymph node, bone marrow, kidney, placenta, or fetus tissues. In one embodiment, a sample comprises a fluid sample, such as peripheral blood, lymph fluid, ascites, serous fluid, pleural effusion, sputum, cerebrospinal fluid, amniotic fluid, lacrimal fluid, stool, or urine. In another embodiment, a sample comprises primary or metastatic NSCLC cells. In yet another embodiment, a sample comprises blood. MicroRNA is readily detectable in blood and blood compartments such as serum or plasma by a number of methods. (Chen X et al, Cell Research 18 983-984, October 2008).

Provided are kits that facilitate assessing the expression of a target. Such kits contain one or more reagents that indicate the presence of a target. Contents of such kits include one or more of the following alone or in combination: one or more oligonucleotide primers capable of hybridizing to sequences within the target which are further optimized for use in a PCR based method, an antisense probe to all or part of target sequence, a ligand with specificity to the target mRNA, protein or other measurable gene product, a label, a buffer, or any other reagent that is useful in a method that assesses the expression of a target whether known or yet to be disclosed.

### EXAMPLES

### Example 1

Expression of a microRNA (miR) with specificity to the 3′UTR of VEGFR2 or VEGFR3 is capable of binding the UTR and silencing the VEGFR2 or VEGFR3 (VEGFR 2/3) expression. Positive expression of such miR indicates reduced VEGFR2/3 expression. Tumors with reduced VEGFR2/3 expression are resistant to VEGFR 2/3 specific tyrosine kinase inhibitors. Conversely, if a tumor displays reduced expression of a miR capable of downregulating VEGFR-2/3, then the result is a more robust VEGFR-2/3 expression, indicating that the tumor is more sensitive to VEGFR2/3 specific tyrosine kinase inhibitors. Assessing VEGFR2/3 expression by miR has advantages over assessing VEGFR2/3 protein directly. Expression by miR is assessed quickly by PCR and high throughput sequencing methods. Further, miR is available in blood and the expression of an individual miR is easily assessed in plasma, serum, or other blood fractions. Such assays allow easy presymptomatic surveillance of a number of diseases, especially cancer.

Search of a publicly available database revealed that miRs, based on their sequence, potentially regulate VEGFR-2/3 expression (Targetscan Database, Whitehead Institute for Biomedical Research, 2006-2008). This comprised searching the 3′ UTR of both genes for potential miR binding sites. One core predicted binding site GCTGCT was common for both the VEGFR2 and VEGFR3 3′UTR's. Using seed sequences, miR-497 was identified as possibly capable of regulating VEGFR2/3. MiR-497 is located on chromosome 17p. The fact that allelic loss in chromosome 17p is frequent in lung cancer (Tonon et al., Proc Natl Acad Sci USA 102:9625-9630, 2005) further led to the selection of miR-497. Allelic loss of 17p occurs at a 50% rate in lung cancer. Additionally, chromosome 17p is situated in close proximity (<1 MB) to the TP53 gene locus, a frequent site of loss of heterozygosity in cancer generally (Chmara et al., Anticancer Res 24:4259-4263, 2004).

MiR-497 expression, VEGFR2/VEGFR3 protein and mRNA expression, and sensitivity of specific tyrosine kinase inhibitor sunitinib to the VEGFR2/3 were assessed in six cell lines derived from non-small cell lung cancer (H1703, A549, H520, H322C, H358, and H157) and are summarized in Table 1 (below).

TABLE 1

| | | | H520 | | | |
|---|---|---|---|---|---|---|
| | | | (Squamous | H322C | H358 | H157 |
| | H1703 | A549 | Cell | (Bronchioloalveolar | (Bronchioloalveolar | (Squamous Cell |
| Data | (Adenocarcinoma) | (Adenocarcinoma) | Carcinoma) | Carcinoma) | Carcinoma) | Carcinoma) |
| microRNA-497 | High | High | High | Low | Low | Low |
| expression | | | | | | |
| VEGFR2 mRNA | Up/Up | NC/Down | Up/Up | Down/Up | NC/Up | NC/Up |
| level (I/M) | | | | | | |
| VEGFR3 mRNA | NC/Up | Down/Down | Up/NC | Down/Up | Up/Down | NC/Up |
| level (I/M) | | | | | | |

I/M = Inhibitor/Mimic,
NC = no change from control

100741 Positive expression of miR-497 is seen in the H1703, A549, and H520 cell lines, while reduced expression is seen in H322C, H358, and H157. The table further shows changes in VEGFR2 and VEGFR3 mRNA expression after transfection with miR-497 inhibitor(I) or miR-497 mimic (M) (an RNA with a similar sequence to that of miR-497) normalized to the miR-497 expression with a control miR in which all three had been normalized to act in mRNA expression.

Transfection of miR-497 mimic and inhibitor (an RNA with a sequence similar to the anti sense sequence of miR-497) into the indicated cell lines was performed, followed by Western blotting and qRT-PCR. Densitometry measurements were taken from Western blot images using Scion Image.

Table 2 summarizes the expression of VEGFR2 protein and VEGFR3 protein in the six listed above cell lines as the IC₅₀ of each cell line to sunitinib (below).

TABLE 2

| Data | H1703 | H520 | H322C | H358 | A549 | H157 |
|---|---|---|---|---|---|---|
| Histology | Adenocarcinoma | Squamous Cell | Bronchioloalveolar | Bronchioloalveolar | Adenocarcinoma | Squamous Cell |
| VEGFR2 | Present | Absent | Present | Present | Present | Present |
| protein | | | | | | |
| VEGFR3 | Absent | Absent | Present | Present | Present | Present |
| protein | | | | | | |
| IC₅₀ | <0.5 μM | 1-3 μM | 9-10 μM | >10 μM | >10 μM | >10 μM |

The first two rows of Table 2 summarize the expression of VEGFR2 protein and VEGFR3 protein in the six listed cell lines by Western blot. The term “present” indicates positive expression of VEGFR2 or VEGFR3 protein.

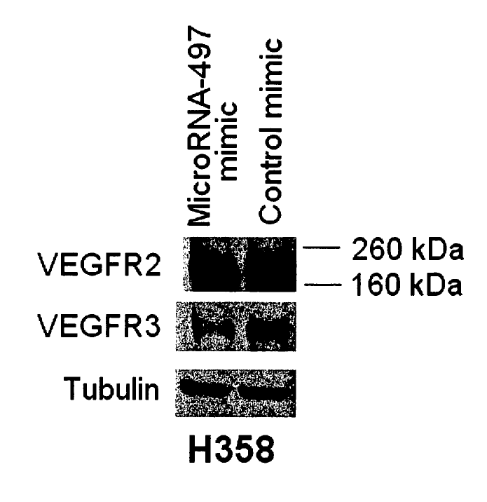

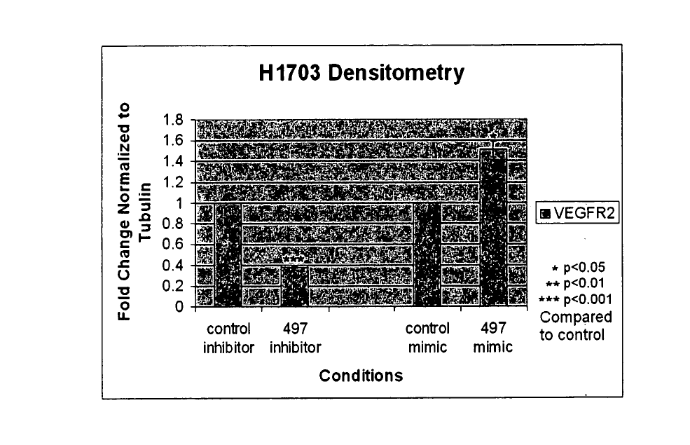

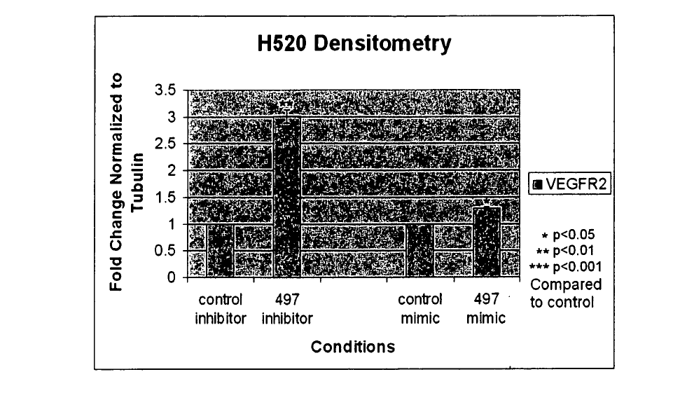

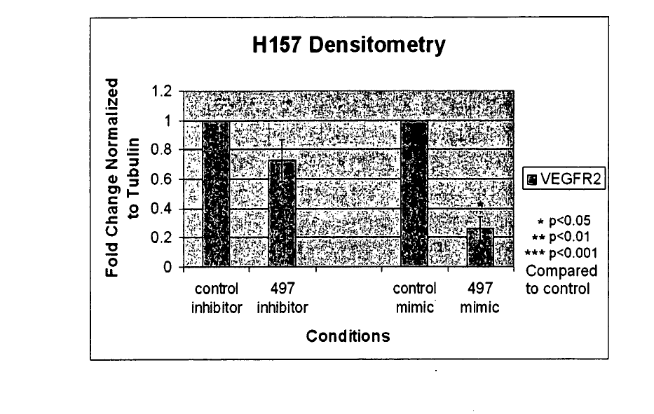

As shown in FIGS. 1 to 5, reduced expression of the VEGFR2 protein with transfection of miR-497 mimic was observed in four of the six tested cell lines. Reduced expression of the VEGFR2 mRNA was not observed in any of the tested cell lines transfected with miR-497 mimic. Increased expression of the VEGFR2 protein was observed in three of the six lines transfected with miR-497 inhibitor. Increased expression of the VEGFR2 mRNA was observed in two of the six cell lines transfected with the miR-497 inhibitor.

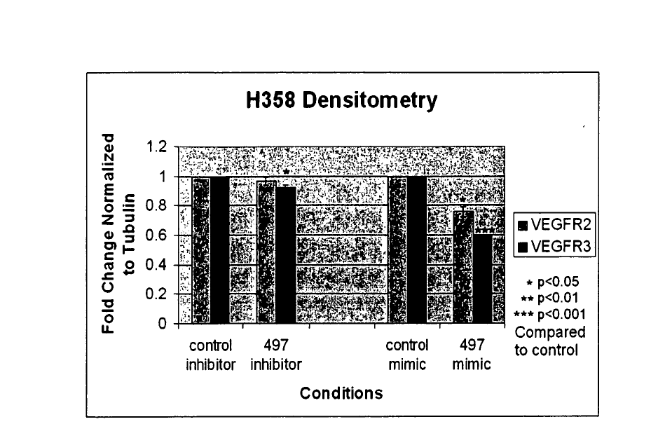

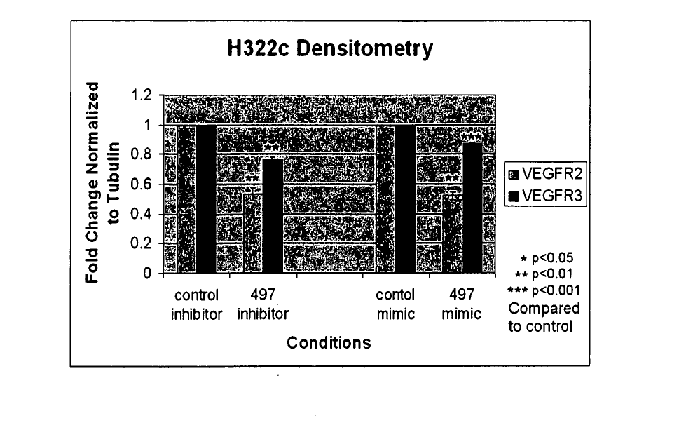

As shown in FIGS. 2 and 7, reduced expression of the VEGFR3 protein was observed with transfection of miR-497 mimic in one of the two cell lines in which it was assessed. Reduced expression of the VEGFR3 message was observed with transfection of miR-497 mimic in one of the six tested cell lines. Increased expression of the VEGFR3 protein was not observed in either of the cell lines transfected with the miR-497 inhibitor in which the VEGFR3 protein expression was assessed. Increased VEGFR3 message was observed in two of the six cell lines when those lines were transfected with the miR-497 inhibitor.

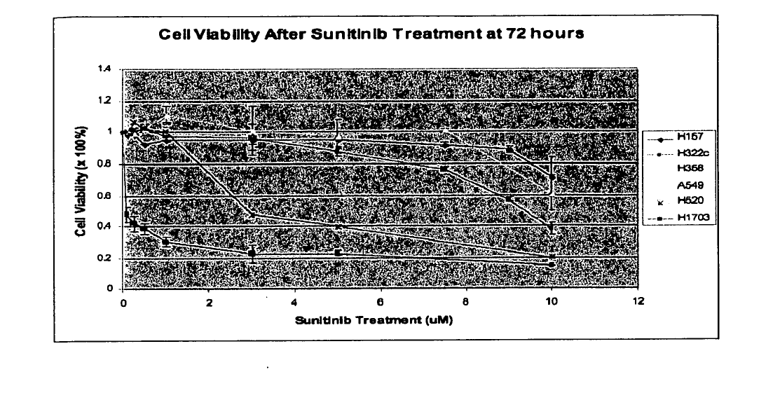

As shown in Table 2 and FIG. 8, untransfected cell lines H1703 and H520 were sensitive to sunitinib, displayed positive expression of miR-497 and both lacked the VEGFR3 protein expression. Additionally, H520 lacked the VEGFR2 protein expression. Untransfected cell lines A549, H322C, H358, and H157 were resistant to sunitinb and displayed positive expression of both VEGFR2 and VEGFR3. Of these, only A549 displayed positive expression of miR-497. In general, in vitro exposure increased sensitivity to VEGFR-2/3 sunitinib correlated with a positive expression of microRNA-497.

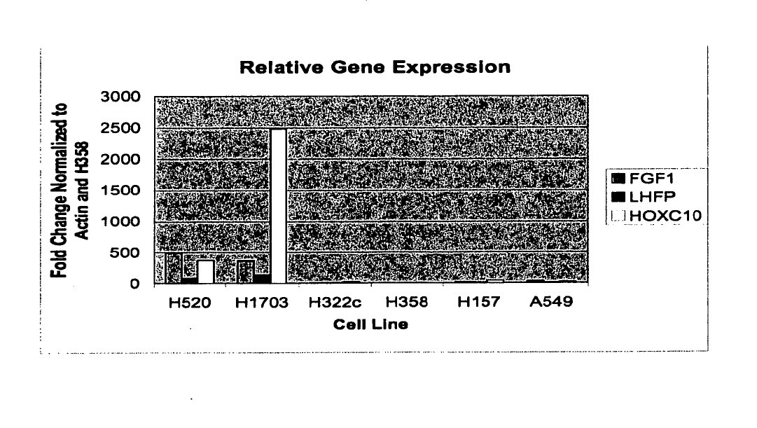

As shown in FIG. 9, microarray gene expression data from NCBI's GEO GSE 4342 were normalized by ‘per chip normalization’ and ‘per gene normalization’ using GeneSpring between resistant and sensitive NSCLC lines. Genes that were identified as having statistically significant differences (p<0.01) when grouped as sunitinib resistant (defined solely for the purposes of this example as having IC50>9 μM) and sunitinib sensitive (defined solely for the purposes of this example as having an IC50<3 μM), were validated by qRT-PCR. Genes meeting those criteria were confirmed by RTPCR. Using qRT-PCR, it was revealed that FGF1 (SEQ ID NO:4), HOXC10 (SEQ ID NO:5), and LHFP (SEQ ID NO:6) were present in NSCLC lines and were resistant to sunitinib. Surprisingly, pathway analysis revealed that these 3 genes were not part of canonical pathways of resistance to sunitinib.

Sequences corresponding to SEQ ID NO:4, SEQ ID NO:5 and SEQ ID NO:6 are provided below.

FGF 1
(SEQ ID NO: 4)
agctgcagta gcctggaggt tcagagagcc gggctactct
gagaagaaga caccaagtgg attctgcttc ccctgggaca
gcactgagcg agtgtggaga gaggtacagc cctcggccta
caagctcttt agtcttgaaa gcgccacaag cagcagctgc
tgagccatgg ctgaagggga aatcaccacc ttcacagccc
tgaccgagaa gtttaatctg cctccaggga attacaagaa
gcccaaactc ctctactgta gcaacggggg ccacttcctg
aggatccttc cggatggcac agtggatggg acaagggaca
ggagcgacca gcacattcag ctgcagctca gtgcggaaag
cgtgggggag gtgtatataa agagtaccga gactggccag
tacttggcca tggacaccga cgggctttta tacggctcac
agacaccaaa tgaggaatgt ttgttcctgg aaaggctgga
ggagaaccat tacaacacct atatatccaa gaagcatgca
gagaagaatt ggtttgttgg cctcaagaag aatgggagct
gcaaacgcgg tcctcggact cactatggcc agaaagcaat
cttgtttctc cccctgccag tctcttctga ttaaagagat
ctgttctggg tgttgaccac tccagagaag tttcgagggg
tcctcacctg gttgacccaa aaatgttccc ttgaccattg
gctgcgctaa cccccagccc acagagcctg aatttgtaag
caacttgctt ctaaatgccc agttcacttc tttgcagagc
cttttacccc tgcacagttt agaacagagg gaccaaattg
cttctaggag tcaactggct ggccagtctg ggtctgggtt
tggatctcca attgcctctt gcaggctgag tccctccatg
caaaagtggg gctaaatgaa gtgtgttaag gggtcggcta
agtgggacat tagtaactgc acactatttc cctctactga
gtaaacccta tctgtgattc ccccaaacat ctggcatggc
tcccttttgt ccttcctgtg ccctgcaaat attagcaaag
aagcttcatg ccaggttagg aaggcagcat tccatgacca
gaaacaggga caaagaaatc cccccttcag aacagaggca
tttaaaatgg aaaagagaga ttggattttg gtgggtaact
tagaaggatg gcatctccat gtagaataaa tgaagaaagg
gaggcccagc cgcaggaagg cagaataaat ccttgggagt
cattaccacg ccttgacctt cccaaggtta ctcagcagca
gagagccctg ggtgacttca ggtggagagc actagaagtg
gtttcctgat aacaagcaag gatatcagag ctgggaaatt
catgtggatc tggggactga gtgtgggagt gcagagaaag
aaagggaaac tggctgaggg gataccataa aaagaggatg
atttcagaag gagaaggaaa aagaaagtaa tgccacacat
tgtgcttggc ccctggtaag cagaggcttt ggggtcctag
cccagtgctt ctccaacact gaagtgcttg cagatcatct
ggggacctgg tttgaatgga gattctgatt cagtgggttg
ggggcagagt ttctgcagtt ccatcaggtc ccccccaggt
gcaggtgctg acaatactgc tgccttaccc gccatacatt
aaggagcagg gtcctggtcc taaagagtta ttcaaatgaa
ggtggttcga cgccccgaac ctcacctgac ctcaactaac
ccttaaaaat gcacacctca tgagtctacc tgagcattca
ggcagcactg acaatagtta tgcctgtact aaggagcatg
attttaagag gctttggccc aatgcctata aaatgcccat
ttcgaagata tacaaaaaca tacttcaaaa atgttaaacc
cttaccaaca gcttttccca ggagaccatt tgtattacca
ttacttgtat aaatacactt cctgcttaaa cttgacccag
gtggctagca aattagaaac accattcatc tctaacatat
gatactgatg ccatgtaaag gcctttaata agtcattgaa
atttactgtg agactgtatg ttttaattgc atttaaaaat
atatagcttg aaagcagtta aactgattag tattcaggca
ctgagaatga tagtaatagg atacaatgta taagctactc
acttatctga tacttattta cctataaaat gagatttttg
ttttccactg tgctattaca aattttcttt tgaaagtagg
aactcttaag caatggtaat tgtgaataaa aattgatgag
agtgttagct cctgtttcat atgaaattga agtaattgtt
aactaaaaac aattccttag taactgaact gtcatattta
gaatggaagg aaaatgacag tttgtgaaag ttcaaagcaa
tagtgcaatt gaagaattga cctaagtaag ctgacattat
ggttaataat agtattttag atttgtgcag caaaataatt
tcataacttt tttgtttttg ttacttggat aagatcaatc
tgttttattt tagtaaatct ttgcaggcaa gttagagaaa
atgcagtgtg gcttaacgtc tctttagtat gaagatttgg
ccagaaaaag atacccagag aggaaatcta agataattat
aatggtccat actttttatt gtatgaatca aactcaagca
taacattggc caaggaaaat taaataccat tgctaacttg
tgaaatggaa gtctgtgatt tcggagatgc aaagcattgt
agtaaaaaca ccaatgtgac ctcgaccatc tcagcccaga
tatcattcat atatctgttc aatgactatt aaggtgccta
ctgtgtgcta ggcactgtac tggatactgg ggaccttgtc
tgtctggttt gctgctgtat cttctcccag ggcattatat
ttatgatgaa agatgctgtg gattcaattc tttcagtcaa
gaataaacac agactttgta ggttcctgct gaataaagca
aatcccagaa acccagattt tggaagaatc agcaacccca
gcataaaata aacccctatc aaaatgtcag aggacatggc
aaggtaaact tagcattttc aactttagaa ccgggtcagc
ttcaggggga ctgctttcaa atcagccaaa gagcctgtca
gatcttctta gaaggaagag gttggtagtt ccctgctctg
ttttgaacat gctctagttt attaacctgg ggacattccc
attgctgtct taagtaagtc tcatagccag ctcctgtcac
gtgactctca tatggattca ttttcgggcc agctctgaac
aaagcatcat gaacatatgt gcttttggtc gtttgcaatg
tgatggtggt ggaggtaggt attggtttcc ttggaaggca
tgataagaaa gattcacaat ggccaacagt gtgtatgaac
aaaaaactga ttggagcatc agctagtact gaaggtcctt
gctttgtgtc agaggcaaag gaacccaagg cgccaagtcc
tcagccttga gtgtactgct gacaactaaa ctcacaggct
gcaaagcaga cctctgatga agatgcctgt tatttcacat
cactgtcttt ttgtgtatca tagtctgcac cttacaaata
ttaataaatg ttccaataat aggtgaaaaa aaaaa
HOXC 10
(SEQ ID NO: 5)
cctcccctcc aaccgcgccc cccctcccgg atggggaaaa
aaaaagatgt cagctcctcc gctgtagtat tgctccttaa
aaacccctct ctctgaaaat gacatgccct cgcaatgtaa
ctccgaactc gtacgcggag cccttggctg cgcccggcgg
aggagagcgc tatagccgga gcgcaggcat gtatatgcag
tctgggagtg acttcaattg cggggtgatg aggggctgcg
ggctcgcgcc ctcgctctcc aagagggacg agggcagcag
ccccagcctc gccctcaaca cctatccgtc ctacctctcg
cagctggact cctggggcga ccccaaagcc gcctatcgcc
tggaacaacc tgttggcagg ccgctgtcct cctgctccta
cccacctagt gtcaaggagg agaatgtctg ctgcatgtac
agcgcagaga agcgggcgaa aagtggcccc gaggcagctc
tctactccca ccccttgccg gagtcctgcc ttggggagca
cgaggtaccc gtgcccagct actaccgcgc cagcccgagc
tactccgcgc tggacaagac gccccactgt tctggggcca
acgacttcga agcccctttc gagcagcggg ccagtctcaa
cccgcgcgcc gaacatctgg aatcgcctca gctggggggc
aaagtgagtt tccctgagac ccccaagtcc gacagccaga
cccccagccc caatgaaatc aagacggagc agagcctggc
gggccctaaa gggagcccct cggagagcga aaaggagagg
gccaaagctg ccgactccag cccagacacc tcggataacg
aagcgaaaga ggagataaag gcagaaaaca ccacaggaaa
ttggctgaca gcaaagagcg gaaggaagaa gaggtgcccc
tatactaaac accagacgct ggaattggag aaagaatttc
tgttcaatat gtatttgacg cgagagcgcc gcctggagat
tagcaagacc attaacctta cagacagaca agtcaaaatc
tggtttcaaa atcgcagaat gaaactcaag aaaatgaacc
gagagaatcg gatccgggaa ctgacctcca attttaattt
cacctgagag cgcggcctct cctcctccct tcccgctcct
tcctctcccc gcccctcctc cctttgtgcc tggtgatata
tttttttttc ctccctgagt ataaatgcaa tgcgactgca
aaaaaggcaa agacctcaga ctctccttcc aagggacctg
tggttcgtgc tgcgaagatg cttccactta aagcatgaga
aatggggtgc cgggatgtgg ggtgtggtgt gtgccctcat
agatgggggt gggagtgtgg ctggtgtgtg tgtcaagccc
tcactcaccc acgcactcac acacagcatt ctgttctcca
tgcaaagtta agatcgaatc catccgcttg taggggaaaa
aaaggaaaaa aattaaccag agagggtctg taatctcgca
gagcacaggc agaatcgttc cttccttgct gcatttcctc
cttagactaa tagacgtttt ggaaagttcg gctagtgttc
gtgtgtttgt cgtagcaccc agagcctcca ccaaaccctc
tccatgtctt tacctcccag tcgctctaag aatctgcttg
aagtctcgta tttgtactgc tttctgcttt tctcccaccc
ctcctagcac ccccacatcc cccatctagt aacatctcag
aaatttcatc cagaggaaca aaaaaattaa aaatagaaca
tagcaaagca aagacagaat gccccccccc ccaaatattg
tcctgtccct gtctgggagt tgtgttattt aaagatattc
tgtatgttgt atcttttgca tgtagcttcc ttaatggaga
aaaaaaaacc taataaattt ccagaatcat aatcctcaaa
aaaaaaaaaa aaaaaaaaaa aaaaaaaaaa aaaaaaaaaa
aaaaaaaaaa aaaaaaa
LHFP
(SEQ ID NO: 6)
aggaggcggt gcgtgcctcg cctgccaaag ggagatccgc
tcctctgcgt gcgatccccg gcgcccgcgc gcgcccacag
cgctccgcca gagctgccgc cgcggactcg ccgggagtgg
gggtctccgc tggtgccagc ccgcttctgg agaccctccg
cctcctgcca acccctgctc ttccaggtcg ggccccgggg
ttctgcggct gttagggaca gaggcaaaga agggcaggac
ggtccggttt cccgtggatg ttcccgcccg agaaagacag
caagttgtgt gtgcgcccgg gacgcgggag ggaaggtagc
cgccgcccgc cagccatgga ccatcatctt tagtgcagag
gatggaaagt tgatgcccag taagactgaa gatccattct
gcattacgga actgtggatt atctgtgggt ccctggtgat
ttcacacctt cattcactcc tgcagtccct gaacacttac
ttggggtcct cattgcccta tctggtgaaa gatggcatcc
agcctgactt gtactggagt aatctgggct ttgctgtctt
ttctttgtgc tgccacctcc tgcgtggggt tctttatgcc
ttactggctc tggggatcac agctgggcaa gcctgtgtcc
ttcggtacct tccggaggtg ctcatatcct gtgcatgatg
agagtcggca gatgatggtg atggtggagg aatgtgggcg
ctatgcctcc ttccagggca tccccagcgc agaatggagg
atctgcacca tagtgaccgg cctgggttgt ggcctcctcc
tcctggtggc gctcactgcc ctcatgggtt gctgtgtttc
cgacctcatc tccaggacag tgggaagagt ggctggagga
attcagtttc ttgggggctt gttgattggt gctggctgtg
ccctctaccc cttgggctgg gacagtgagg aagtccggca
gacttgtggc tacacttctg gccagtttga cctggggaag
tgtgaaatcg gctgggccta ctactgcacg ggagcaggtg
ccactgccgc catgctgctg tgcacgtggc tggcttgctt
ttcgggcaag aaacagaagc actacccata ctgagatgga
gctaccaaga gcagacagag gagaagatgg gccaaagggg
cttggagagg tcaaaacatc cacctacctt caaaaggtgg
gatagtagtt ctaatccaat acaatgctaa taaaatgaaa
cccgataaaa tcaggaacat gatataggaa ggaaggattg
taggagattt gtgggggaaa aaaaaggaga gtatagaatg
atggagaaaa atggaccaaa ggctaaaaat attgcagggc
atcgggtgtt tctattccac agagtattgt taatgtacaa
cacacacaca cacacacaca cacacacaca cacacacaca
cacacaacaa atctacatat acaaacaagg gtttgggttt
tagttttttt tttttaaggt gaggactcag aaaatcaaag
ggctagtaga aacagtgtta tgttgggaag cagggtaccc
ccaaagatgt tccctgtagg tcacggcact cccaaaagca
cacaagcaca tacagacata tgcatcccca cacacgccta
tgcacaaacg tggattatcg cacagactgg gaggtttagt
ggtgcatttc tcctctgttt tctttttaat atacatttaa
aatacagtat tatcacttta taaaacatac attaagccta
ataaatggac caataagcca aactatcagt attttgtata
tcctgcataa actctaattt agttcctcaa catattttca
gtgtttatgc agacctttag agttaagcct ttgtatttcc
atgttattcc acaatatgca atatttctct gagtagcttc
tgctatgata ttcttatgaa gaaaaggggc aactttctgt
ccactatagg agagaattca ccattattgt actgtgctgt
accacattta tttctatatt cattttgtaa aaaatttaaa
agtgctattt tgtttgtatt tgaaaatctc tgtgaataaa
ttctctcttt gatcaataaa aaaaaaaaaa aaaaaaaaaa

All publications and patent applications cited in this application are herein incorporated by reference as if each individual publication or patent application were specifically and individually indicated to be incorporated by reference. Although the foregoing invention has been described in some detail by way of illustration and example for purposes of clarity and understanding, it will be readily apparent to those of ordinary skill in the art in light of the teachings of this invention that certain changes and modifications made be made thereto without departing from the spirit or scope of the appended claims.

1. A method of evaluating sensitivity of a cancer cell to a tyrosine kinase inhibitor comprising:
assessing the expression of SEQ ID NO:1 in a sample comprising a gene product; and
correlating reduced expression of SEQ ID NO:1 with sensitivity to the tyrosine kinase inhibitor.

2. The method of claim 1, wherein the sample comprises a tumor biopsy or a blood.

3. (canceled)

4. The method of claim 1, wherein the gene product comprises mRNA.

5. The method of claim 4 wherein assessing the expression of SEQ ID NO:1 comprises using RTPCR, microarray, or Northern Blot.

6-7. (canceled)

8. The method of claim 1, wherein the tyrosine kinase inhibitor inhibits VEGFR2 (SEQ ID NO:2).

9. The method of claim 1, wherein the tyrosine kinase inhibitor inhibits VEGFR3 (SEQ ID NO. 3).

10. The method of claim 1, wherein the tyrosine kinase inhibitor is sunitinib.

11. The method of claim 1, wherein the tyrosine kinase inhibitor is sorafenib.

12. The method of claim 1, wherein the cancer cell is a non-small cell lung cancer cell.

13. A method of slowing the expansion of a cancer cell in a subject comprising:
assessing the expression of SEQ ID NO:1 in a sample;
correlating reduced expression of SEQ ID NO:1 with sensitivity to a tyrosine kinase inhibitor; and
administering an effective amount of the tyrosine kinase inhibitor to the subject.

14. The method of claim 13, wherein the sample is a human sample.

15. The method of claim 14, wherein the sample comprises a blood fraction or a tumor biopsy.

16. (canceled)

17. The method of claim 13, wherein the cancer cell comprises a loss of heterozygosity in chromosomal region 17p.

18. The method of claim 13, wherein the cancer cell is a non-small cell lung cancer cell.

19. The method of claim 18, wherein the tyrosine kinase inhibitor is sunitinib.

20. A kit that facilitates the assessment of the expression of miR-497 comprising a reagent capable of specifically recognizing miR-497 or a product thereof.

21. The kit of claim 20, wherein the reagent comprises an oligonucleotide or antisense nucleic acid.

22. (canceled)

23. The kit of claim 20, wherein the reagent is bound to a solid support.

24. The kit of claim 20, further comprising a fluorescent label.

25. The kit of claim 20, to further comprising a reagent capable of specifically recognizing a second gene or a product thereof.

26. A method of evaluating the sensitivity of a cancer cell to a tyrosine kinase inhibitor comprising:
assessing the expression of a target gene product selected from the group consisting of FGF1 (SEQ ID NO:4), HOXC10 (SEQ ID NO:5) and LHFP (SEQ ID NO:6) in a sample; and
correlating positive expression of the target gene product with resistance to the tyrosine kinase inhibitor.

27. The method of claim 26, wherein the sample comprises a tumor biopsy or blood.

28. (canceled)

29. The method of claim 26, wherein the cancer cell is a non-small cell lung cancer cell.

30. The method of claim 26, wherein the gene product comprises mRNA.

31. The method of claim 30, wherein assessing the expression of the gene product comprises using RTPCR, microarray, or Northern Blot.

32-33. (canceled)

34. The method of claim 26, wherein the gene product comprises protein.

35. The method of claim 34, wherein assessing the expression of the gene product comprises using a ligand capable of specifically recognizing the gene product.

36. The method of claim 35, wherein the ligand comprises an antibody.

37. The method of claim 36, wherein assessing the expression of the gene product comprises using immunohistochemical methods, ELISA, flow cytometry, or mass spectrometry.

38-40. (canceled)

41. The method of claim 26, wherein the cancer cell is a non-small cell lung cancer cell.

42. The method of claim 41, wherein the tyrosine kinase inhibitor is sunitinib.

43. A method of slowing the expansion of a cancer cell in a subject comprising:
assessing the expression of a target selected from the group consisting of FGF1 (SEQ ID NO:4), HOXC10 (SEQ ID NO:5) and LHFP (SEQ ID NO:6) in a sample;
correlating positive expression of the target with sensitivity to a tyrosine kinase inhibitor; and
administering an effective amount of the tyrosine kinase inhibitor to the subject.

44. The method of claim 43, wherein the sample is a human sample.

45. The method of claim 44, wherein the sample comprises a blood fraction or a tumor biopsy.

46. (canceled)

47. The method of claim 43, wherein the cancer cell is a non-small cell lung cancer cell.

48. The method of claim 47, wherein the tyrosine kinase inhibitor is sunitinib.

49. A kit that facilitates the assessment of the expression of a target selected from the group consisting of FGF1 (SEQ ID NO:4), HOXC10 (SEQ ID NO:5) and LHFP (SEQ ID NO:6) comprising a reagent capable of specifically recognizing a target selected from the group consisting of FGF1 (SEQ ID NO:4), HOXC10 (SEQ ID NO:5) and LHFP (SEQ ID NO:6) or a gene product thereof.

50. The kit of claim 49, wherein the reagent comprises an oligonucleotide or an antisense nucleic acid.

51. (canceled)

52. The kit of claim 49, wherein the reagent comprises an antibody.

53. The kit of claim 49, wherein the reagent is bound to a solid support.

54. The kit of claim 49, further comprising a fluorescent label.

55. The kit of claim 49, further comprising a reagent capable of specifically recognizing a second gene or a product thereof.


SEQUENCE LISTING (6 sequences)

SEQ ID NO: 1 - RNA, 21 nt, Homo sapiens
cagcagcacacugugguuugu

SEQ ID NO: 2 - DNA, 1686 bp, Homo sapiens
gcagcatcggacaagacccccagcacttgggggttcaggcccggcagggcgggcagagggctggaggcccaggctgggaactcatctggttgaactctggtggcacaggagtgtcctcttccctctctgcagacttcccagctaggaagagcaggactccaggcccaaggctcccggaattccgtcaccacgactggccagggccacgctccagctgccccggcccctccccctgagattcagatgtcatttagttcagcatccgcaggtgctggtcccggggccagcacttccatgggaatgtctctttggcgacctcctttcatcacactgggtggtggcctggtccctgttttcccacgaggaatctgtgggtctgggagtcacacagtgttggaggttaaggcatacgagagcagaggtctcccaaacgccctttcctcctcaggcacacagctactctccccacgagggctggctggcctcacccacccctgcacagttgaagggaggggctgtgtttccatctcaaagaaggcatttgcagggtcctcttctgggcctgaccaaacagccaactagcccctggggtggccaccagtatgacagtattatacgctggcaacacagaggcagcccgcacacctgcgcctgggtgttgagagccatcctgcaagtctttttcaacagaacttcacagactgttagagctgctgagaagaatttgctttccgaattcagcctggaaggcgcccagggacagctgtactgagtctagatgactctgacccccgccccaggtcaaggccagcagagcagtcagtgcctctggagaaggcccttgctctcccacctggcccagactccgaggagcctgggtctggagctgccggtctggttcttccctttagagcccggatctgccacctgcggcccctcccaagccgtgaaccagctcatgagagatgaacactgtgggatccactcaggaaggctcggggctggcacaaaggaccacccagcattgccctgtgccacccagcactcagtggacattctggggacctgccttcagccttttcctgccctgtgcctgacatcagcaccctggctggtcagaatgccgccctcccagaggagcagccgagagatcccctgaaggctggaggcattctgctcaggacccctatcccagctcacagtgcccaaccatctcaccaggagaaagagccacatccccacgttaggaccacggagactgaccaccaccctgaccccccaaacccacgcaccagacgcttgcaggacaggcgccgcgcagcgggcaggggcttgcccggccgaccctcccctccccacctcccccactgcgcgttactccaggatatgccgagtgcacgtataaggtcatcttcgtcgtccccgtggacctcccccttcctctgcacgtcgtccaacgtgggactggcgtgtcaggcttccctgggaggatctggaggttgttctctgcagagaaccagcctggctcctggcgcgcacctctgctcccttctcctcactacccacccacgcatgtaccgggaaaaaaactactatgcccttctagaccatgttctgagaaaagatcgaaaatatttaacaagagataataataaatctgatgccggtctttgtgtgtgttgcgga

SEQ ID NO: 3 - DNA, 1686 bp, Homo sapiens; identical to SEQ ID NO: 2

SEQ ID NO: 4 - DNA, 3755 bp, Homo sapiens
agctgcagtagcctggaggttcagagagccgggctactctgagaagaagacaccaagtggattctgcttcccctgggacagcactgagcgagtgtggagagaggtacagccctcggcctacaagctctttagtcttgaaagcgccacaagcagcagctgctgagccatggctgaaggggaaatcaccaccttcacagccctgaccgagaagtttaatctgcctccagggaattacaagaagcccaaactcctctactgtagcaacgggggccacttcctgaggatccttccggatggcacagtggatgggacaagggacaggagcgaccagcacattcagctgcagctcagtgcggaaagcgtgggggaggtgtatataaagagtaccgagactggccagtacttggccatggacaccgacgggcttttatacggctcacagacaccaaatgaggaatgtttgttcctggaaaggctggaggagaaccattacaacacctatatatccaagaagcatgcagagaagaattggtttgttggcctcaagaagaatgggagctgcaaacgcggtcctcggactcactatggccagaaagcaatcttgtttctccccctgccagtctcttctgattaaagagatctgttctgggtgttgaccactccagagaagtttcgaggggtcctcacctggttgacccaaaaatgttcccttgaccattggctgcgctaacccccagcccacagagcctgaatttgtaagcaacttgcttctaaatgcccagttcacttctttgcagagccttttacccctgcacagtttagaacagagggaccaaattgcttctaggagtcaactggctggccagtctgggtctgggtttggatctccaattgcctcttgcaggctgagtccctccatgcaaaagtggggctaaatgaagtgtgttaaggggtcggctaagtgggacattagtaactgcacactatttccctctactgagtaaaccctatctgtgattcccccaaacatctggcatggctcccttttgtccttcctgtgccctgcaaatattagcaaagaagcttcatgccaggttaggaaggcagcattccatgaccagaaacagggacaaagaaatccccccttcagaacagaggcatttaaaatggaaaagagagattggattttggtgggtaacttagaaggatggcatctccatgtagaataaatgaagaaagggaggcccagccgcaggaaggcagaataaatccttgggagtcattaccacgccttgaccttcccaaggttactcagcagcagagagccctgggtgacttcaggtggagagcactagaagtggtttcctgataacaagcaaggatatcagagctgggaaattcatgtggatctggggactgagtgtgggagtgcagagaaagaaagggaaactggctgaggggataccataaaaagaggatgatttcagaaggagaaggaaaaagaaagtaatgccacacattgtgcttggcccctggtaagcagaggctttggggtcctagcccagtgcttctccaacactgaagtgcttgcagatcatctggggacctggtttgaatggagattctgattcagtgggttgggggcagagtttctgcagttccatcaggtcccccccaggtgcaggtgctgacaatactgctgccttacccgccatacattaaggagcagggtcctggtcctaaagagttattcaaatgaaggtggttcgacgccccgaacctcacctgacctcaactaacccttaaaaatgcacacctcatgagtctacctgagcattcaggcagcactgacaatagttatgcctgtactaaggagcatgattttaagaggctttggcccaatgcctataaaatgcccatttcgaagatatacaaaaacatacttcaaaaatgttaaacccttaccaacagcttttcccaggagaccatttgtattaccattacttgtataaatacacttcctgcttaaacttgacccaggtggctagcaaattagaaacaccattcatctctaacatatgatactgatgccatgtaaaggcctttaataagtcattgaaatttactgtgagactgtatgttttaattgcatttaaaaatatatagcttgaaagcagttaaactgattagtattcaggcactgagaatgatagtaataggatacaatgtataagctactcacttatctgatacttatttacctataaaatgagatttttgttttccactgtgctattacaaattttcttttgaaagtaggaactcttaagcaatggtaattgtgaataaaaattgatgagagtgttagctcctgtttcatatgaaattgaagtaattgttaactaaaaacaattccttagtaactgaactgtcatatttagaatggaaggaaaatgacagtttgtgaaagttcaaagcaatagtgcaattgaagaattgacctaagtaagctgacattatggttaataatagtattttagatttgtgcagcaaaataatttcataacttttttgtttttgttacttggataagatcaatctgttttattttagtaaatctttgcaggcaagttagagaaaatgcagtgtggcttaacgtctctttagtatgaagatttggccagaaaaagatacccagagaggaaatctaagataattataatggtccatactttttattgtatgaatcaaactcaagcataacattggccaaggaaaattaaataccattgctaacttgtgaaatggaagtctgtgatttcggagatgcaaagcattgtagtaaaaacaccaatgtgacctcgaccatctcagcccagatatcattcatatatctgttcaatgactattaaggtgcctactgtgtgctaggcactgtactggatactggggaccttgtctgtctggtttgctgctgtatcttctcccagggcattatatttatgatgaaagatgctgtggattcaattctttcagtcaagaataaacacagactttgtaggttcctgctgaataaagcaaatcccagaaacccagattttggaagaatcagcaaccccagcataaaataaacccctatcaaaatgtcagaggacatggcaaggtaaacttagcattttcaactttagaaccgggtcagcttcagggggactgctttcaaatcagccaaagagcctgtcagatcttcttagaaggaagaggttggtagttccctgctctgttttgaacatgctctagtttattaacctggggacattcccattgctgtcttaagtaagtctcatagccagctcctgtcacgtgactctcatatggattcattttcgggccagctctgaacaaagcatcatgaacatatgtgcttttggtcgtttgcaatgtgatggtggtggaggtaggtattggtttccttggaaggcatgataagaaagattcacaatggccaacagtgtgtatgaacaaaaaactgattggagcatcagctagtactgaaggtccttgctttgtgtcagaggcaaaggaacccaaggcgccaagtcctcagccttgagtgtactgctgacaactaaactcacaggctgcaaagcagacctctgatgaagatgcctgttatttcacatcactgtctttttgtgtatcatagtctgcaccttacaaatattaataaatgttccaataataggtgaaaaaaaaaa

SEQ ID NO: 5 - DNA, 2017 bp, Homo sapiens
cctcccctccaaccgcgccccccctcccggatggggaaaaaaaaagatgtcagctcctccgctgtagtattgctccttaaaaacccctctctctgaaaatgacatgccctcgcaatgtaactccgaactcgtacgcggagcccttggctgcgcccggcggaggagagcgctatagccggagcgcaggcatgtatatgcagtctgggagtgacttcaattgcggggtgatgaggggctgcgggctcgcgccctcgctctccaagagggacgagggcagcagccccagcctcgccctcaacacctatccgtcctacctctcgcagctggactcctggggcgaccccaaagccgcctatcgcctggaacaacctgttggcaggccgctgtcctcctgctcctacccacctagtgtcaaggaggagaatgtctgctgcatgtacagcgcagagaagcgggcgaaaagtggccccgaggcagctctctactcccaccccttgccggagtcctgccttggggagcacgaggtacccgtgcccagctactaccgcgccagcccgagctactccgcgctggacaagacgccccactgttctggggccaacgacttcgaagcccctttcgagcagcgggccagtctcaacccgcgcgccgaacatctggaatcgcctcagctggggggcaaagtgagtttccctgagacccccaagtccgacagccagacccccagccccaatgaaatcaagacggagcagagcctggcgggccctaaagggagcccctcggagagcgaaaaggagagggccaaagctgccgactccagcccagacacctcggataacgaagcgaaagaggagataaaggcagaaaacaccacaggaaattggctgacagcaaagagcggaaggaagaagaggtgcccctatactaaacaccagacgctggaattggagaaagaatttctgttcaatatgtatttgacgcgagagcgccgcctggagattagcaagaccattaaccttacagacagacaagtcaaaatctggtttcaaaatcgcagaatgaaactcaagaaaatgaaccgagagaatcggatccgggaactgacctccaattttaatttcacctgagagcgcggcctctcctcctcccttcccgctccttcctctccccgcccctcctccctttgtgcctggtgatatatttttttttcctccctgagtataaatgcaatgcgactgcaaaaaaggcaaagacctcagactctccttccaagggacctgtggttcgtgctgcgaagatgcttccacttaaagcatgagaaatggggtgccgggatgtggggtgtggtgtgtgccctcatagatgggggtgggagtgtggctggtgtgtgtgtcaagccctcactcacccacgcactcacacacagcattctgttctccatgcaaagttaagatcgaatccatccgcttgtaggggaaaaaaaggaaaaaaattaaccagagagggtctgtaatctcgcagagcacaggcagaatcgttccttccttgctgcatttcctccttagactaatagacgttttggaaagttcggctagtgttcgtgtgtttgtcgtagcacccagagcctccaccaaaccctctccatgtctttacctcccagtcgctctaagaatctgcttgaagtctcgtatttgtactgctttctgcttttctcccacccctcctagcacccccacatcccccatctagtaacatctcagaaatttcatccagaggaacaaaaaaattaaaaatagaacatagcaaagcaaagacagaatgcccccccccccaaatattgtcctgtccctgtctgggagttgtgttatttaaagatattctgtatgttgtatcttttgcatgtagcttccttaatggagaaaaaaaaacctaataaatttccagaatcataatcctcaaaaaaaaaaaaaaaaaaaaaaaaaaaaaaaaaaaaaaaaaaaaaaaaaaaaaaaaaaaa

SEQ ID NO: 6 - DNA, 2120 bp, Homo sapiens
aggaggcggtgcgtgcctcgcctgccaaagggagatccgctcctctgcgtgcgatccccggcgcccgcgcgcgcccacagcgctccgccagagctgccgccgcggactcgccgggagtgggggtctccgctggtgccagcccgcttctggagaccctccgcctcctgccaacccctgctcttccaggtcgggccccggggttctgcggctgttagggacagaggcaaagaagggcaggacggtccggtttcccgtggatgttcccgcccgagaaagacagcaagttgtgtgtgcgcccgggacgcgggagggaaggtagccgccgcccgccagccatggaccatcatctttagtgcagaggatggaaagttgatgcccagtaagactgaagatccattctgcattacggaactgtggattatctgtgggtccctggtgatttcacaccttcattcactcctgcagtccctgaacacttacttggggtcctcattgccctatctggtgaaagatggcatccagcctgacttgtactggagtaatctgggctttgctgtcttttctttgtgctgccacctcctgcgtggggttctttatgccttactggctctggggatcacagctgggcaagcctgtgtccttcggtaccttccggaggtgctcatatcctgtgcatgatgagagtcggcagatgatggtgatggtggaggaatgtgggcgctatgcctccttccagggcatccccagcgcagaatggaggatctgcaccatagtgaccggcctgggttgtggcctcctcctcctggtggcgctcactgccctcatgggttgctgtgtttccgacctcatctccaggacagtgggaagagtggctggaggaattcagtttcttgggggcttgttgattggtgctggctgtgccctctaccccttgggctgggacagtgaggaagtccggcagacttgtggctacacttctggccagtttgacctggggaagtgtgaaatcggctgggcctactactgcacgggagcaggtgccactgccgccatgctgctgtgcacgtggctggcttgcttttcgggcaagaaacagaagcactacccatactgagatggagctaccaagagcagacagaggagaagatgggccaaaggggcttggagaggtcaaaacatccacctaccttcaaaaggtgggatagtagttctaatccaatacaatgctaataaaatgaaacccgataaaatcaggaacatgatataggaaggaaggattgtaggagatttgtgggggaaaaaaaaggagagtatagaatgatggagaaaaatggaccaaaggctaaaaatattgcagggcatcgggtgtttctattccacagagtattgttaatgtacaacacacacacacacacacacacacacacacacacacacacacacacaacaaatctacatatacaaacaagggtttgggttttagttttttttttttaaggtgaggactcagaaaatcaaagggctagtagaaacagtgttatgttgggaagcagggtacccccaaagatgttccctgtaggtcacggcactcccaaaagcacacaagcacatacagacatatgcatccccacacacgcctatgcacaaacgtggattatcgcacagactgggaggtttagtggtgcatttctcctctgttttctttttaatatacatttaaaatacagtattatcactttataaaacatacattaagcctaataaatggaccaataagccaaactatcagtattttgtatatcctgcataaactctaatttagttcctcaacatattttcagtgtttatgcagacctttagagttaagcctttgtatttccatgttattccacaatatgcaatatttctctgagtagcttctgctatgatattcttatgaagaaaaggggcaactttctgtccactataggagagaattcaccattattgtactgtgctgtaccacatttatttctatattcattttgtaaaaaatttaaaagtgctattttgtttgtatttgaaaatctctgtgaataaattctctctttgatcaataaaaaaaaaaaaaaaaaaaaaaa